(19) United States
(12) Patent Application Publication
(10) Pub. No.: US 2011/0281943 A1
(43) Pub. Date: Nov. 17, 2011

(54) Topical Composition Comprising Ascorbic Acid Derivatives

(75) Inventors: Pei Min Lee, Taipei City (TW); Leon Hides, North Parramatta (AU); Jen Han Lee, Taipei City (TW); Man Li Peng, Taipei City (TW); Lin Chiao Chen, Taipei City (TW)

(73) Assignee: CORUM INC., Taipei City (TW)

(21) Appl. No.: 13/097,976

(22) Filed: Apr. 29, 2011

Related U.S. Application Data
(60) Provisional application No. 61/334,028, filed on May 12, 2010.

(51) Int. Cl. A61K 8/49; A61Q 90/00

(52) U.S. Cl. 514/474

(57) ABSTRACT

The present invention discloses topical composition comprising ascorbic acid derivatives. The inventive topical composition that the ascorbic acid derivatives is homogeneously incorporated therein can make the ascorbic acid derivatives more widely used as active ingredient in skin topical formulation.

### BACKGROUND OF THE INVENTION

1. Field of the Invention

The invention is generally related to skin topical composition, especially to topical composition comprising ascorbic acid derivatives.

2. Description of the Prior Art

L-ascorbic acid is widely used in many cosmetic formulations, because L-ascorbic acid is a well-known water-soluble antioxidant that has whitening effect and serves as a cofactor of prolinehydroxylase to promote synthesis of collage (Quaglino, D. Jr., et al., J. Biol. Chem., p 272-345, 1997). L-ascorbic acid is also used in various products requiring a long-term anti-oxidation effect. But L-ascorbic acid is not so reliable because it is sensitive to heat, light, and air. Because of the notorious instability of L-ascorbic acid, many attempts have been made to develop more stable compounds.

According to U.S. Pat. No. 7,741,496 B2, it is disclosed that the novel ascorbic acid derivatives therein are structurally stabile ascorbic acid and effective whitening agent.

A skin topical cosmetic formulation can produced as aqueous phase or emulsion product. For instance, the cosmetic product is skin serum, lotion, or cream. In order to make the mentioned ascorbic acid derivatives efficiently penetrating skin as an active ingredient, it is an important subject to provide a composition that the ascorbic acid derivatives can be homogeneously incorporated into a formulation.

### SUMMARY OF THE INVENTION

In view of the above background, topical composition comprising ascorbic acid derivatives is disclosed, wherein the ascorbic acid derivatives is homogeneously incorporated into the topical composition. The mentioned ascorbic acid derivatives compound has a general formula as following:

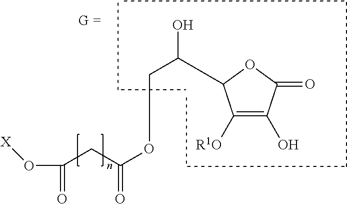

wherein X is selected from the group consisting one of the following: the G group, hydrogen, linear alkyl moiety, branched alkyl moiety, cyclic alkyl moiety; n ranges from 2 to 12; and R¹ is selected from the group consisting one of the following: hydrogen, alkyl group having 1 to 4 carbon, linear alkyl moiety, branched alkyl moiety.

### DESCRIPTION OF THE PREFERRED EMBODIMENTS

What is probed into the invention is a topical composition comprising ascorbic acid derivatives. Detail descriptions of the structure and elements will be provided as followed in order to make the invention thoroughly understood. The application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail as followed. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In one embodiment of the present invention, a topical composition comprising ascorbic acid derivatives is disclosed. The mentioned topical composition comprises ascorbic acid derivatives, and a solvent. The mentioned ascorbic acid derivatives has a general formula as following:

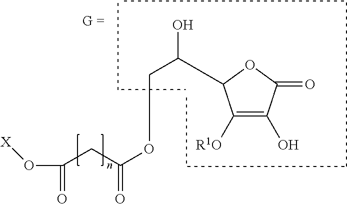

Wherein X is selected from the group consisting one of the following: the G group, hydrogen, linear alkyl moiety, branched alkyl moiety, cyclic alkyl moiety. In the mentioned general formula, n ranges from 2 to 12. R¹ of the mentioned general formula is selected from the group consisting one of the following: hydrogen, alkyl group having 1 to 4 carbon, linear alkyl moiety, branched alkyl moiety. According to this embodiment, the amount of the ascorbic acid derivatives is about 0.001 to 10% by weight of the total weight of the topical composition.

In one preferred example of this embodiment, the formula of the mentioned ascorbic acid derivatives is

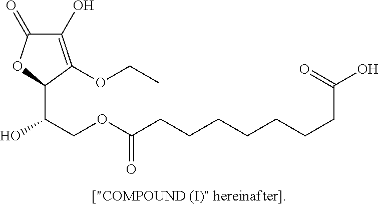

In another preferred example of this embodiment, the formula of the mentioned ascorbic acid derivatives is

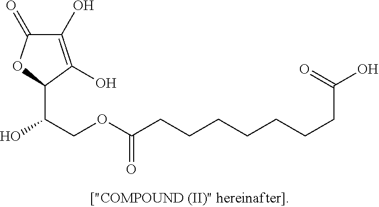

The mentioned ascorbic acid derivatives are mostly in the form of white crystalline powders. In order to incorporate the ascorbic acid derivatives within a formulation as a more active ingredient, sometimes it is necessary for the ascorbic acid derivatives to be present as solutions as the solid form is incapable of penetrating the skin. The mentioned solvent of the topical composition of this embodiment is employed for transferring the ascorbic acid derivatives into solutions. So that the solutions can subsequently form the basis of the aqueous phase of various formulations. In one preferred example of this embodiment, the mentioned solvent is polar. In another preferred example of this embodiment, the mentioned solvent is selected from at least one of the group consisted of the following: water, alcohol, ether, di-ol, poly-ol. In another preferred example of this embodiment, the solvent is selected from at least one of the group consisting of the following: alcohol, glycol, propylene glycol, butylene glycol, glycerol, PEG-8, dipropylene glycol, glycol ether, polyethylene glycol with molecular weight ranging from 200 to 6000, polypropylene glycol with molecular weight ranging from 200 to 6000.

When the composition according to this embodiment, the composition according to this embodiment can further comprises different ingredients when the composition is employed for different skin-topical formulations.

### EXAMPLES

Examples of formulations of the topical compositions according to the present invention will be given. The following are the best compositions thereof.

### Example 1

### Formulation for Herbal Whitening Serum

| | | % |
|---|---|---|
| phase | Formulation | by weight |
| A | Water | to 100.00 |
| A | Microcrystalline Cellulose (and) | 0.80 |
| | Cellulose Gum | |
| A | Xanthan Gum | 0.40 |
| B | Octoxynol-11 (and) Polysorbate 20 | 0.30 |
| B | Fragrance | 0.10 |
| B | Phenoxyethanol | 0.40 |
| C | Water | 10.00 |
| C | Sodium Citrate | 1.27 |
| C | Citric Acid | 0.78 |
| C | Sodium Bisulfite | 0.15 |
| C | COMPOUND(I) | 2.00 |
| D | Jojoba Wax PEG-120 Esters | 2.00 |
| D | Sclerotium Gum | 2.00 |
| D | Methylisothiazolinone | 0.10 |

Preparation Method of this Formulation:

Firstly, the components of Phase A, Phase B, Phase C were mixed respectively. The microcrystalline cellulose and xanthan gum was homogeneously dispersed in the distilled water (phase A), while sodium citrate, citric acid, sodium bisulfite and the COMPOUND (I) were dissolved in distilled water (phase C). The Phase B and Phase C were added into Phase A, and then the mixture of Phase A, Phase B, and Phase C were mixed homogeneously. Subsequently, the rest ingredients (Phase D) were added into the above-mentioned mixture and mixed.

### Example 2

### Formulation for Skin Rejuvenation and Brightening Lotion

| | | |
|---|---|---|
| | | % |
| phase | Formulation | by weight |

(Oil phase)

| | | |
|---|---|---|
| A | Cetyl Alcohol (and) Glyceryl Stearate | 4.00 |
| | (and) PEG-75 Stearate (and) | |
| | Steareth-20 | |
| A | Cetearyl Alcohol | 2.00 |
| A | Dioctyl Sebacate | 4.00 |
| A | Ethylhexyl Isononanoate | 3.00 |
| A | Macadamia Integrifolia Nut Oil | 1.00 |
| A | Dimethicone | 2.00 |

(Aqueous phase)

| | | |
|---|---|---|
| B | Water | 31.60 |
| B | Microcrystalline Cellulose (and) | 1.50 |
| | Cellulose Gum | |
| C | Water | to 100.00 |
| C | Disodium EDTA | 0.05 |
| C | Citric Acid | 0.63 |
| C | Sodium Citrate | 1.27 |
| D | COMPOUND (I) | 2.00 |
| D | Water | 4.00 |
| D | Sodium Bisulfite | 0.20 |
| E | Glycerin (and) Palmitoyl Tripeptide-1 | 3.00 |
| E | Sodium Hyaluronate (1%) | 1.00 |
| E | Methylisothiazolinone | 0.10 |
| E | Fragrance | 0.15 |

Preparation Method of this Formulation:

The oil phase and aqueous phase are separately heated and held at 80° C. The oil phase was added to the aqueous phase and the resultant mixture emulsified by homomixer and cooled to 40° C. The COMPOUND (I) was added to water, and then Sodium Bisulfite was added therein. The rest of the ingredients were added, while stirring well.

### Example 3

### Formulation for Whitening and Moisturizing Cream

| | | |
|---|---|---|
| | | % |
| phase | Formulation | by weight |

(Oil phase)

| | | |
|---|---|---|
| A | Cetyl Alcohol (and) Glyceryl Stearate | 6.00 |
| | (and) PEG-75 Stearate (and) Steareth-20 | |
| A | Cetearyl Alcohol | 1.20 |
| A | Ethylhexyl Isononanoate | 4.00 |
| A | Simmondsia Chinensis (Jojoba) Seed Oil | 1.00 |
| A | PPG-3 Benzyl Ether Myristate | 0.55 |
| A | Cholesteryl Hydroxystearate | 0.30 |
| A | Helianthus Annuus (Sunflower) Seed Oil | 3.00 |
| A | Dimethicone | 3.00 |

(Aqueous phase)

| | | |
|---|---|---|
| B | Water | to 100.00 |
| B | Citric Acid | 0.21 |
| B | Sodium Citrate | 1.82 |
| B | Disodium EDTA | 0.05 |
| B | Glycerin | 3.00 |
| C | COMPOUND (I) | 1.00 |
| C | Water | 5.00 |
| C | Butylene glycol | 4.00 |
| C | Sodium Bisulfite | 0.15 |
| D | Glycerin (and) Palmitoyl Hexapeptide-12 | 1.20 |
| D | Dimethicone (and) Dimethiconol | 3.00 |
| D | Sodium Hyaluronate | 1.00 |
| D | Methylisothiazolinone | 0.10 |
| D | Fragrance | 0.10 |

Preparation Method of this Formulation:

The oil phase and aqueous phase are separately heated and held at 75° C. The oil phase was added to the aqueous phase and the resultant mixture emulsified by homomixer and cooled to 40° C. The COMPOUND (I) was added to water, and then Sodium Bisulfite was added therein. The rest of the ingredients were added, while stirring well.

### Example 4

### Formulation for Whitening and Moisture Spray

| | | % |
|---|---|---|
| phase | Formulation | by weight |
| A | Glycerin | 3.00 |
| A | Hydrolyzed jojoba esters (and) water | 1.00 |
| A | Octoxynol-11 (and) Polysorbate 20 | 1.20 |
| A | Alcohol | 3.00 |
| A | Water | to 100.00 |
| A | Citric Acid | 0.80 |
| A | Sodium Citrate | 1.30 |
| A | Sodium Bisulfite | 0.20 |
| A | Methylisothiazolinone | 0.10 |
| B | Alcohol | 5.00 |
| B | COMPOUND (II) | 2.00 |
| C | Fragrance | 0.10 |
| C | Octoxynol-11 (and) Polysorbate 20 | 0.30 |

Preparation Method of this Formulation:

Each of the phase A, phase B and phase C was separately prepared by low shear mixing. Phase B and phase C were added into phase A, and the mixture of phase A, phase B, and phase C were mixed at room temperature to form an uniform solution.

### Example 5

### Formulation for Whitening Sunscreen

| | | |
|---|---|---|
| | | % |
| phase | Formulation | by weight |

(Oil phase)

| | | |
|---|---|---|
| A | Bis-Ethylhexyloxyphenol | 1.80 |
| | Methoxyphenyl Triazine | |
| A | Ethylhexyl Methoxycinnamate | 7.50 |
| A | Lauryl/Myristyl Benzoate | 5.80 |
| A | Ethylhexyl Isononanoate | 2.00 |
| A | Octocrylene | 1.00 |
| A | Ceteary Alcohol | 1.00 |
| A | Myristyl Myristate | 1.20 |
| A | Macadamia Integrifolia Nut Oil | 1.00 |
| A | Dimethicone | 1.50 |
| | Cetyl Alcohol (and) Glyceryl | |
| A | Stearate (and) PEG-75 Stearate (and) | 6.00 |
| | Ceteth-20 (and) Steareth-20 | |

(Aqueous phase)

| | | |
|---|---|---|
| B | Water | to 100.00 |
| B | Citric Acid | 0.50 |
| B | Sodium Citrate | 1.00 |
| B | Glycerin | 3.00 |
| C | Water | 5.00 |
| C | COMPOUND (II) | 2.00 |
| C | Disodium EDTA | 0.05 |
| C | Sodium Bisulfite | 0.10 |
| D | Sclerotium Gum | 1.00 |
| D | Water | 1.00 |
| E | Glycerin (and)Palmitoyl Dipeptide-10 | 2.00 |
| E | Phenoxyethanol | 0.50 |
| E | Methylisothiazolinone | 0.08 |
| E | Fragrance | 0.20 |

Preparation Method of this Formulation:

The oil phase and aqueous phase are separately heated and held at 80° C. The oil phase was added to the aqueous phase and the resultant mixture was emulsified by homomixer and cooled to 40° C. The COMPOUND (II) was added to water, and then Sodium Bisulfite was added therein. Subsequently, the rest of the ingredients were added therein while stirring well.

To sum up, the present invention discloses topical composition comprising ascorbic acid derivatives. According to this present invention, the ascorbic acid derivatives can be homogeneously incorporated into the topical composition. So that when the topical composition is used in cosmetic formulation, the ascorbic acid derivatives can be as an active and reliable ingredient with better skin penetration.

Other modifications and variations are possibly developed in light of the above demonstrations. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A topical composition, comprising:
an ascorbic acid derivatives; and
a solvent;
wherein said ascorbic acid derivatives has a general formula as following: 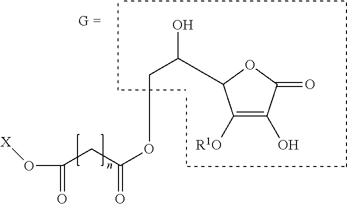
wherein X is selected from the group consisting one of the following: the G group, hydrogen, linear alkyl moiety, branched alkyl moiety, cyclic alkyl moiety;
n ranges from 2 to 12; and
R¹ is selected from the group consisting one of the following:
hydrogen, alkyl group having 1 to 4 carbon, linear alkyl moiety, branched alkyl moiety.

2. The topical composition according to claim 1, wherein the formula of said ascorbic acid derivatives is

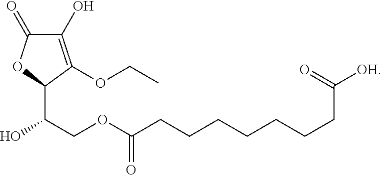

3. The topical composition according to claim 1, wherein the formula of said ascorbic acid derivatives is

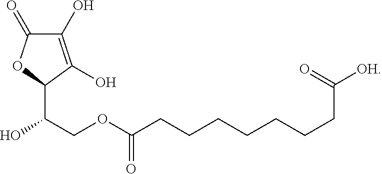

4. The topical composition according to claim 1, wherein the solvent is polar.

5. The topical composition according to claim 1, wherein the solvent is selected from at least one of the group consisting of the following: water, alcohol, ether, di-ol, poly-ol.

6. The topical composition according to claim 1, wherein the solvent is selected from at least one of the group consisting of the following: alcohol, glycol, propylene glycol, butylene glycol, glycerol, PEG-8, dipropylene glycol, glycol ether, polyethylene glycol with molecular weight ranging from 200 to 6000, polypropylene glycol with molecular weight ranging from 200 to 6000.

7. The topical composition according to claim 1, wherein the amount of the ascorbic acid derivatives is about 0.001 to 10% by weight of the total weight of the topical composition.
